(19) United States
(12) Patent Application Publication
(10) Pub. No.: US 2022/0211380 A1
(43) Pub. Date: Jul. 7, 2022

(54) SYSTEMS, DEVICES, AND METHODS FOR DIAGNOSING AMYLOIDOSIS

(71) Applicant: Alleviant Medical, Inc., Austin, TX (US)

(72) Inventor: Thomas D. PATE, Austin, TX (US)

(21) Appl. No.: 17/569,383

(22) Filed: Jan. 5, 2022

Related U.S. Application Data
(60) Provisional application No. 63/134,822, filed on Jan. 7, 2021.

(51) Int. Cl. A61B 17/11; A61M 60/148; A61N 1/36; A61B 18/14; A61M 60/857

(52) U.S. Cl. CPC A61B 17/11; A61M 60/148; A61B 2018/00577; A61B 18/1492; A61M 60/857; A61N 1/36114

(57) ABSTRACT

Disclosed herein are methods, systems, and devices for diagnosing and treating heart failure. In some variations, a method of treating a patient may comprise forming an anastomosis between a right atrium and a left atrium of the patient by removing a portion of an interatrial septum. The removed portion of the interatrial septum may be analyzed for a cardiac amyloidosis diagnosis.

### CROSS-REFERENCE TO RELATED APPLICATIONS

This application claims the benefit of U.S. Provisional Application No. 63/134,822, filed Jan. 7, 2021, the content of which is hereby incorporated by reference in its entirety.

### TECHNICAL FIELD

Devices, systems, and methods herein relate to diagnosing heart disease, including but not limited to diagnosing cardiac amyloidosis.

### BACKGROUND

Congestive heart failure (CHF) is marked by declining function of the heart muscle, either due to a weakening of its pumping ability or a stiffening of the muscle with decreased ability to fill with blood prior to ejection. With poor flow of blood from the heart to vital organs, the renin-angiotensin-aldosterone system (RAAS) is activated, which signals the body to retain fluid, thereby increasing pressure in the heart chambers. In particular, as the left atrial pressure (LAP) rises, fluid backs up into the pulmonary circulation and may lead to pulmonary edema and severe shortness of breath. As such, additional devices, systems, and methods for diagnosing and treating heart disease may be desirable.

### SUMMARY

Described herein are devices, systems, and methods for one or more of diagnosing and treating heart disease. These devices and systems may form an anastomosis in an anatomical structure and enable biopsy of a portion of interatrial septum tissue to aid diagnosis and treatment of heart disease. In some variations, a method of treating a patient comprises forming an anastomosis between a right atrium and a left atrium of the patient by removing a portion of an interatrial septum, and analyzing the removed portion of the interatrial septum for a cardiac amyloidosis diagnosis.

Also described here are methods of treating a patient comprising removing a portion of an interatrial septum to reduce blood pressure in a left atrium of the patient, diagnosing cardiac amyloidosis based on the removed portion of the interatrial septum, and administering a therapy to the patient based on the cardiac amyloidosis diagnosis.

In some variations, a method may comprise removing a portion of an interatrial septum, and analyzing the removed portion for cardiac amyloidosis.

In some variations, a method of treating a patient may comprise removing a portion of an interatrial septum, diagnosing cardiac amyloidosis based on analyzing the removed portion of the interatrial septum, and administering a therapy to the patient based on the cardiac amyloidosis diagnosis.

In some variations, analyzing the removed portion of the interatrial septum may comprise one or more of gross inspection, histological analysis, electron microscopy, mass spectrometry, and genetic testing. In some variations, analyzing the removed portion of the interatrial septum may comprise analysis for one or more of fibrosis, hypertrophy, atrophy, necrosis, and inflammation. In some variations, the therapy may comprise one or more of a medication, chemotherapy, surgery, tissue ablation, and stent implantation.

In some variations, administering the therapy may comprise administering an effective amount of the medication comprising one or more of an immunosuppressive drug, steroid, tafamidis, diflunisal, patisiran, and inotersen to the patient.

In some variations, removing a portion of right ventricle tissue, and analyzing the removed portion of the right ventricle tissue for the cardiac amyloidosis diagnosis. In some variations, removing the portion of the interatrial septum and administering the therapy treats one or more of heart failure and cardiac amyloidosis. In some variations, removing the portion of the interatrial septum comprises one or more of mechanical cutting and tissue ablation using a tissue capture device. In some variations, the anastomosis may be formed using the tissue capture device. In some variations, tissue ablation may comprise delivering a biphasic radiofrequency waveform to the interatrial septum.

In some variations, the method may further comprise advancing a tissue capture device into a heart of the patient, capturing the portion of the interatrial septum using the tissue capture device, and withdrawing the portion of the interatrial septum and the tissue capture device from the heart. In some variations, the captured portion of the interatrial septum may be disposed in a lumen of the tissue capture device. In some variations, the anastomosis may comprise a diameter of between about 1 mm and about 1.5 cm.

In some variations, removing the portion of the interatrial septum may form an anastomosis between a right atrium and a left atrium of the patient. In some variations, removing the portion of the interatrial septum may reduce blood pressure in a left atrium of the patient.

### BRIEF DESCRIPTION OF THE DRAWINGS

FIG. 1 provides a cross-sectional representation of a heart showing various anatomical structures.

FIG. 2 is a flowchart of an illustrative variation of a method of forming an anastomosis.

FIGS. 3A-3F are schematic perspective views of an illustrative variation of a method of forming an anastomosis using a tissue capture system.

FIGS. 4A and 4B are schematic perspective views of an illustrative variation of a tissue capture device in an endocardial space. FIGS. 4C-4F are schematic cross-sectional side views of illustrative variations of a tissue capture device in an endocardial space.

FIGS. 5A and 5B are side views of an illustrative variation of a tissue capture device in an endocardial space.

FIGS. 6A and 6B are images of an anastomosis formed in porcine tissue.

FIGS. 7A and 7B are perspective views of an illustrative variation of a tissue capture device engaged to cut tissue. FIG. 7C is an image of an anastomosis formed in tissue.

FIGS. 8A and 8B are perspective views of an illustrative variation of a tissue capture device engaged to cut tissue.

FIG. 9 is a schematic block diagram of an illustrative variation of a tissue capture system.

FIG. 10 is a cross-sectional side view of an illustrative variation of a tissue capture device in an open and closed configurations.

### DETAILED DESCRIPTION

Described here are devices, systems, and methods for diagnosing and treating heart failure (e.g., congestive heart failure, heart failure with preserved ejection fraction (HFpEF), cardiac amyloidosis) by reducing blood pressure in a left atrium of a patient and analyzing (e.g., taking a biopsy of) a portion of the interatrial septum. For example, a HFpEF patient may be treated by forming an anastomosis between a right atrium and a left atrium may to relieve elevated left atrial blood pressure by removing a portion of the interatrial septum. The removed portion of the interatrial septum may be captured and analyzed (e.g., histological analysis). An additional therapy may be administered to the patient when the removed interatrial septum tissue comprises cardiac amyloidosis. In this manner, a more accurate diagnosis of a patient's condition may be obtained and additional therapy may be administered to improve patient outcomes. By contrast, conventional diagnosis of amyloidosis requires a separate biopsy procedure to obtain a sample of right ventricular tissue. Thus, the methods described herein may both diagnose and treat heart disease with a fewer number of devices and steps to improve one or more of procedural efficiency, patient safety, and patient outcomes.

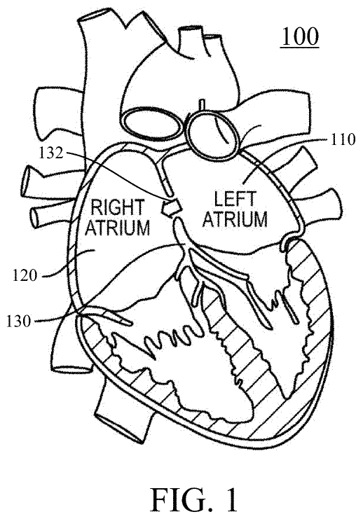

In instances where the heart is the relevant anatomy, it may be helpful to briefly identify and describe the relevant heart anatomy. FIG. 1 is a cross-sectional view of the heart (100). Shown there is the left atrium (110), right atrium (120), and interatrial septum (130). FIG. 1 illustrates an opening (132) (e.g., aperture) formed between the left atrium (110) and the right atrium (120). For example, the opening (132) may be created during an anastomosis procedure using the systems, devices, and methods described herein. The opening (132) may have predetermined characteristics configured to treat heart failure and enable diagnosis of heart disease.

### I. Methods

Also described here are methods of treating a patient using the systems and devices described herein. In particular, the systems, devices, and methods described herein may be used to capture, excise, and remove a predetermined portion of interatrial septum tissue to create an anastomosis for treating heart failure and for diagnosing one or more heart conditions such as amyloidosis.

In some variations, a method of treating heart disease may include advancing a tissue capture device into a heart (e.g., a right atrium of a patient). For example, a guidewire may be advanced across an interatrial septum of the heart and into a left atrium. The tissue capture device may be advanced such that a distal portion of the device is disposed within the right atrium and a proximal portion of the device is disposed in the left atrium. The tissue capture device may comprise a tissue engagement feature (e.g., barb) configured to engage and capture (e.g., hold, secure) tissue. In some variations, a portion of the interatrial septum having a predetermined size may be cut (e.g., excised) from the heart using one or more of mechanical and radiofrequency (RF) energy, thereby forming an anastomosis between the right atrium and the left atrium. The excised tissue may be enclosed by the tissue capture device to prevent tissue loss. The removed portion of the interatrial septum may be analyzed (e.g., biopsied) for heart disease such as cardiac amyloidosis. In some variations, an additional therapy may be administered to the patient in response to the diagnosis of heart disease. In this manner, the anastomosis formed in the heart and administered therapy may synergistically treat the patient without additional steps. Moreover, greater insight into a patient's health may be obtained as a result of analyzing the removed portion of the interatrial septum even if additional therapy is not administered.

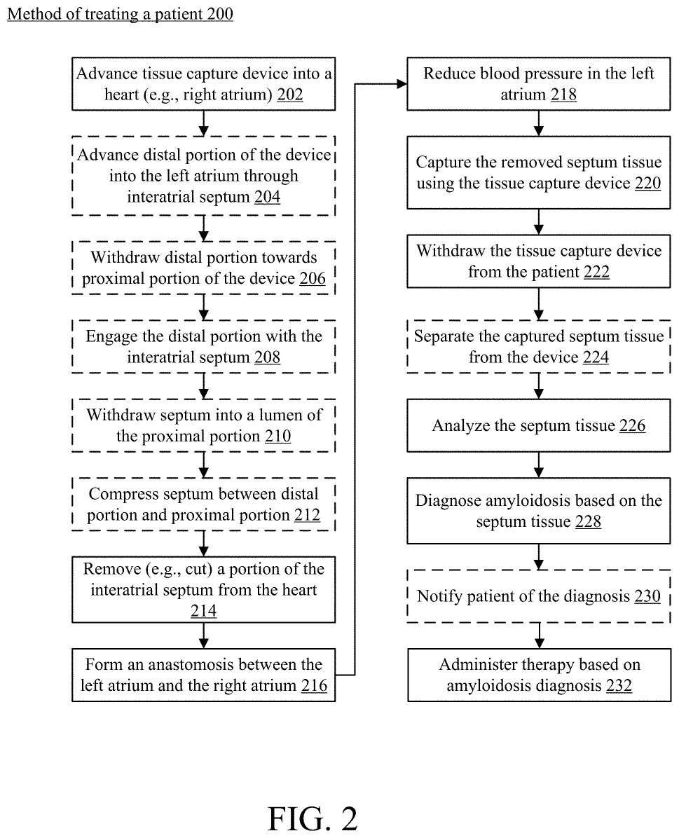

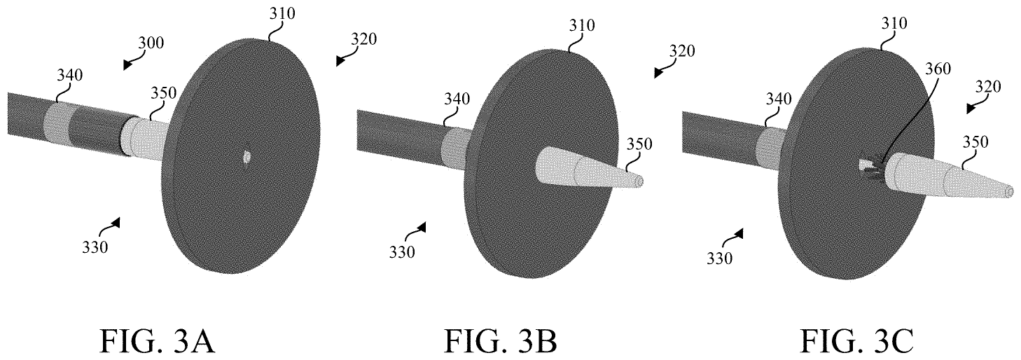

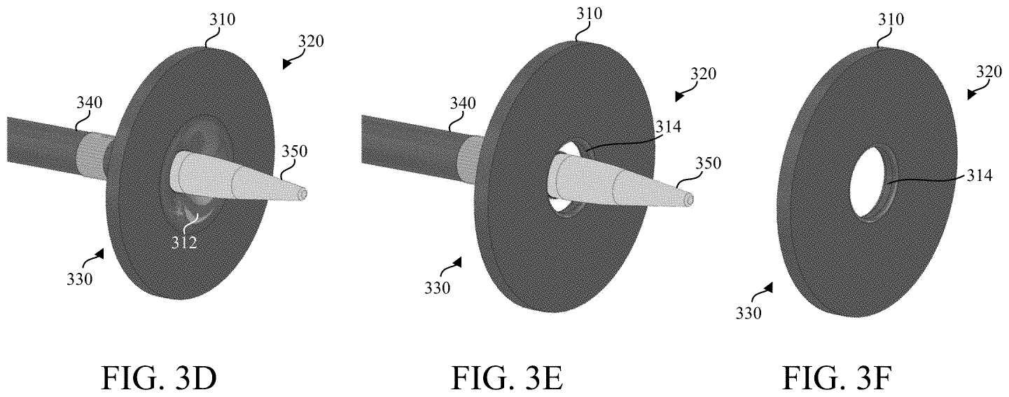

FIG. 2 is a flowchart that generally describes a variation of a method of treating a patient (200). One or more of the steps depicted in FIG. 2 may be performed as shown in the corresponding steps in FIGS. 3A-5B described in more detail herein and using any of the devices described herein. The method (200) may include advancing a tissue capture device into a heart of a patient (202). For example, the tissue capture device may be advanced into a right atrium. In some variations, the tissue capture device may be advanced over a guidewire and inserted through the femoral vein using, for example, a transseptal puncture method. In some variations, the tissue capture device within the right atrium may be oriented approximately perpendicular to an interatrial septum.

In some variations, the tissue capture device may be indirectly visualized as necessary throughout a tissue capture procedure. Indirect visualization, such as echocardiography and/or fluoroscopy, may assist an operator in positioning and/or aligning the tissue capture device relative to tissue. A user may compress and cut a predetermined portion of the interatrial septum.

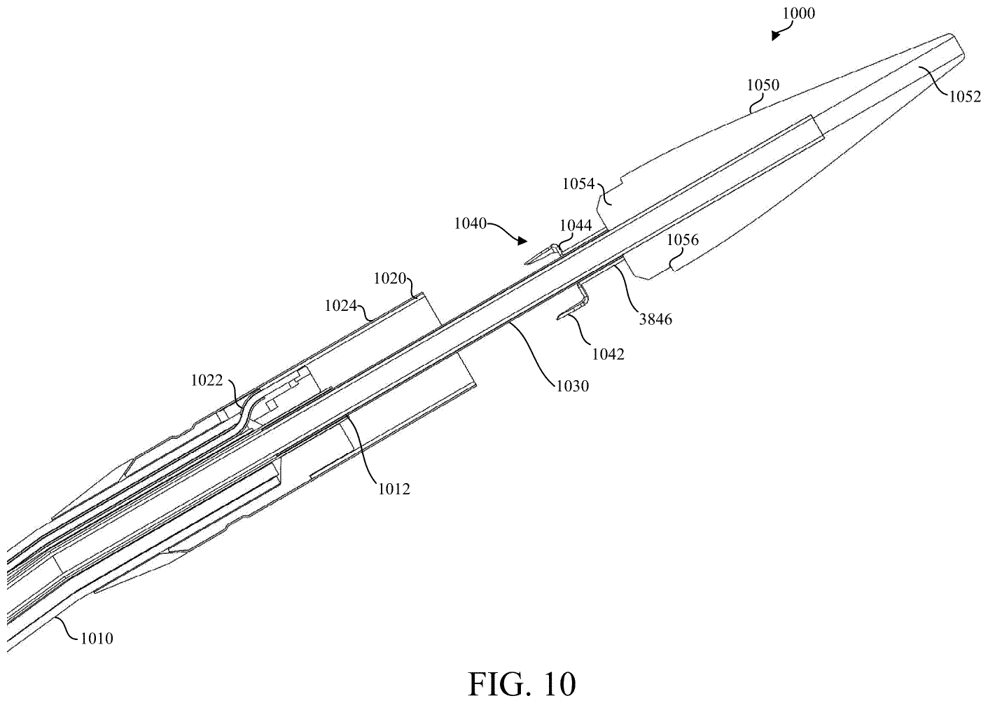

Optionally, a distal portion of the tissue capture device may be advanced into a left atrium through the interatrial septum (204). For example, the distal portion comprising a dilator may be advanced (e.g., over a guidewire) across the interatrial septum such that the guidewire and dilator are located in the left atrium. A tissue engagement feature of the distal portion may be advanced into the left atrium such that a proximal portion of the tissue capture device is located in the right atrium. Septum tissue may slide over the tissue engagement feature as it is advanced into the left atrium. In some variations, the distal portion of the tissue capture device may be configured to translate relative to the proximal portion of the tissue capture device. For example, FIG. 10 depicts a dilator (1050) of a distal portion of tissue capture device (1000) configured to translate relative to an electrode (1020) of a proximal portion of the tissue capture device (1000).

Optionally, the distal portion of the tissue capture device may be withdrawn relative to a proximal portion of the tissue capture device (206). In some variations, the distal portion may be withdrawn while the proximal portion is held in a substantially fixed position in the right atrium. In some variations, indirect visualization may confirm a position of the tissue engagement feature, relative to the interatrial septum tissue.

Optionally, as the distal portion of the tissue capture device is withdrawn, the tissue engagement feature may engage a predetermined portion of the interatrial septum (208).

In some variations, the tissue engagement features described herein may have a configuration designed to engage the septum without shearing the tissue (e.g., breaking or tearing through one or more layers of the interatrial septum) such that the engaged portion of the septum remains intact when engaged to the tissue engagement feature. That is, the forces applied by the tissue engagement features described herein allow the structural integrity of the engaged tissue to be maintained even when the tissue engagement feature pierces through the septum. This may ensure that the engaged portion of the septum to be excised remains held and secured by the tissue engagement feature throughout the procedure, thereby improving the consistency and safety of the methods described herein.

Optionally, the engaged portion of the septum may be withdrawn into a lumen of the proximal portion (210). In some variations, an engaged portion of the interatrial septum may form a tent over the tissue engagement feature as the septum is withdrawn into the lumen of the proximal portion (e.g., a lumen of an electrode as shown in FIG. 10). In this manner, interatrial septum tissue to be cut may be secured within the tissue capture device prior to excision to reduce the risk of uncontrolled tissue loss in the heart chambers and vasculature.

Optionally, the septum may be compressed between the distal portion and the proximal portion of the tissue capture device (212). Tissue compression may reduce the amount of energy (e.g., mechanical and/or electrical) needed to cut the interatrial septum and form an anastomosis. For example, the tissue capture device may be configured to apply a mechanical compression force to the septum tissue.

In some variations, the engaged portion of the interatrial septum may be removed (e.g., cut, captured, excised) from the heart using the tissue capture device (214). Removing the portion of the interatrial septum may comprise one or more of mechanical cutting and tissue ablation using a tissue capture device. For example, a blade may mechanically cut a hole in the interatrial septum. Additionally or alternatively, an ablation waveform may be delivered to an electrode to cut the interatrial septum. For example, a signal generator may generate a biphasic radiofrequency waveform configured to ablate a portion of the interatrial septum held by the tissue capture device. In some variations, the electrode may be configured to transmit about 50 mA to about 4 A of current between about 0.1 kV and about 4.0 kV at a rate of up to about 500 kHz. In some variations, the ablation waveform may comprise a first waveform followed by a second waveform different from the first waveform. The first waveform may comprise a first voltage and the second waveform may comprise a second voltage.

In some variations, the removed portion of the interatrial septum may comprise one or more pieces. For example, the removed portion may be a single contiguous piece or a plurality of pieces. The removed portion may comprise portions from one side or both sides of the interatrial septum. The tissue may be removed using one or more steps, procedures, and/or devices. For example, the same device may be used to perform step (214) multiple times to remove different portions of the interatrial septum. In some variations, a portion of right ventricle tissue may be removed from the heart. In some variations, removing the portion of the interatrial septum may treat one or more of heart failure and cardiac amyloidosis.

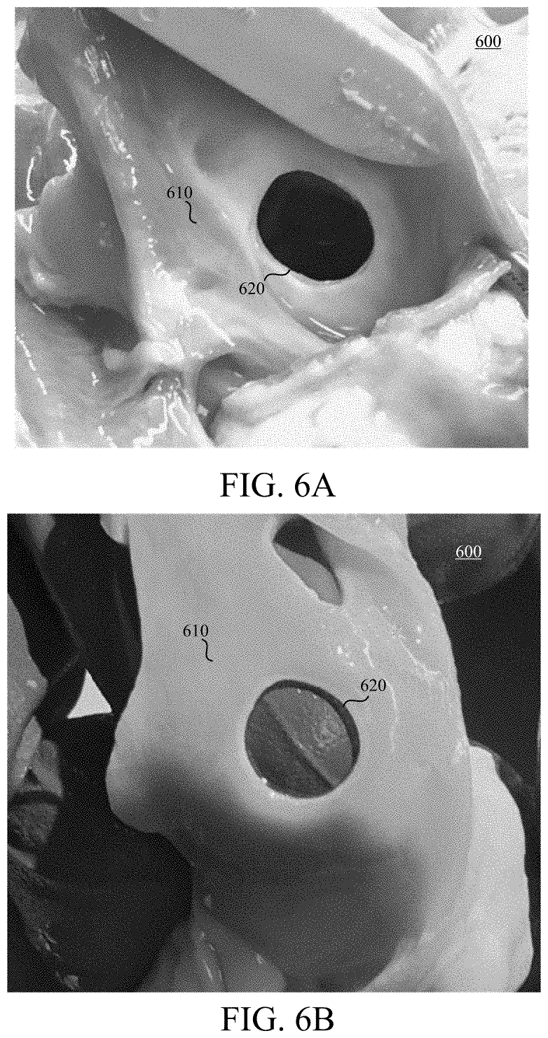

In some variations, an anastomosis may be formed between the left atrium and the right atrium (216). That is, the anastomosis is formed once the engaged portion of the interatrial septum has been removed (e.g., cut, separated) from the heart. In some variations, the anastomosis may be formed using the tissue capture device. In some variations, the anastomosis may comprise a diameter of between about 1 mm and about 1.5 cm. As described in more detail herein, FIGS. 6A and 6B are images (600) of an anastomosis (620) formed in porcine tissue (610).

In some variations, a blood pressure in the left atrium may be reduced in response to the anastomosis (e.g., removing the portion of the interatrial septum) (218).

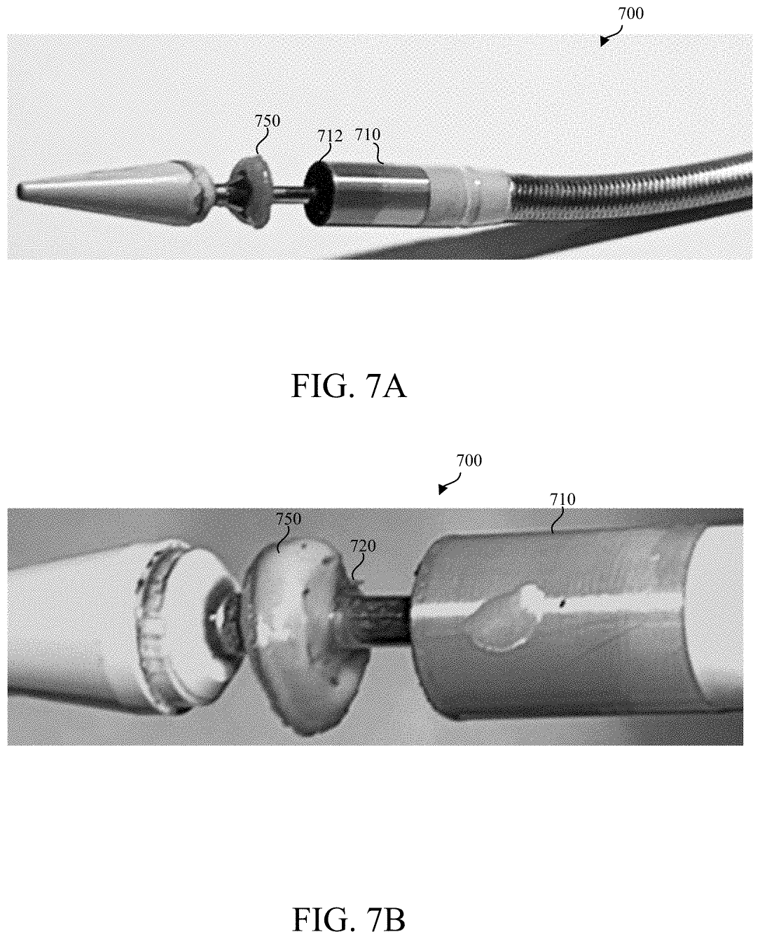

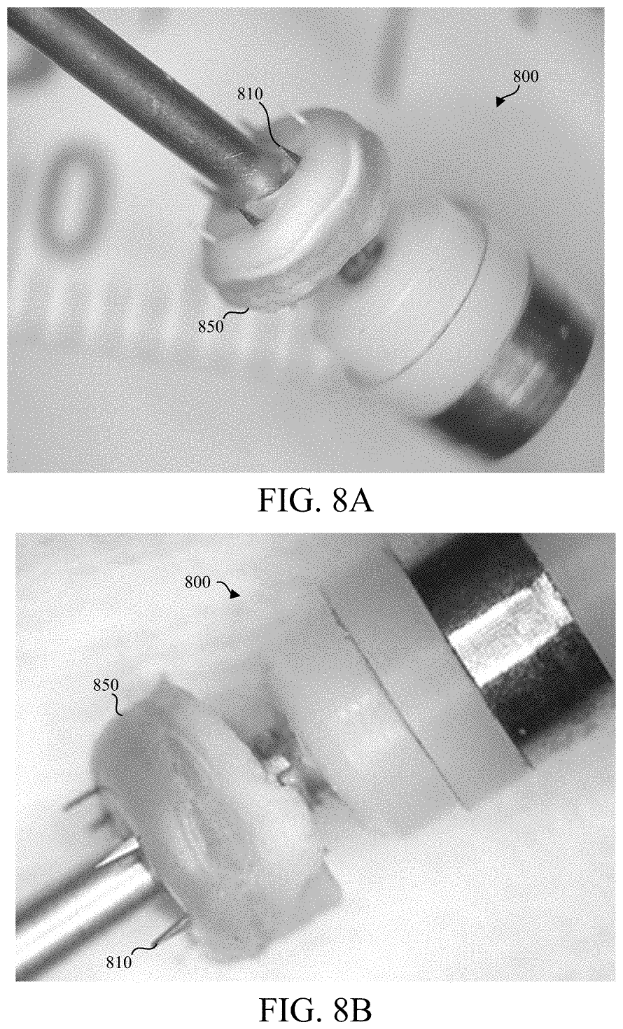

In some variations, the removed septum tissue may be captured by the tissue capture device (220). For example, the captured portion of the interatrial septum may be disposed in (e.g., held and/or secured within) a lumen of the tissue capture device. As described in more detail herein, FIGS. 7A, 7B, 8A, and 8B are images of septum tissue captured by a tissue capture device. The septum tissue may be captured by, for example, a barb and held within a lumen of an electrode.

In some variations, the tissue capture device and captured portion of the interatrial septum may be withdrawn from the heart and patient (222). For example, the captured tissue may be enclosed within a tissue capture device as the device is withdrawn from the body of the patient to ensure that the captured tissue may be analyzed for an amyloidosis diagnosis and to reduce the risk of uncontrolled tissue loss in the heart chambers and vasculature.

In some variations, the captured septum tissue may be separated from a tissue capture device (224). For example, the captured septum tissue may be removed from a barb of a tissue capture device.

In some variations, the captured septum tissue may be analyzed for one or more heart conditions (226). The septum tissue may undergo one or more of histological analysis, mass spectrometry, genetic testing, combinations thereof, and the like. In some variations, analysis of the removed portion of the interatrial septum may comprise analysis for one or more of fibrosis (e.g., myocardial fibrosis), hypertrophy (e.g., myocyte hypertrophy), myocarditis, atrophy, necrosis, inflammation, combinations thereof, and the like. For example, the removed portion of the interatrial septum may be analyzed for cardiac amyloidosis. In some variations, a removed portion of right ventricle tissue may be analyzed for a cardiac amyloidosis diagnosis.

In some variations, the captured tissue may be stained to facilitate histological analysis. Staining may comprise one or more of hematoxylin and eosin, Masson's trichrome, iron, Congo Red, CD68, combinations thereof, and the like. For example, analysis for cardiac amyloidosis may be based on Congo Red staining.

In some variations, amyloidosis (e.g., cardiac amyloidosis) may be diagnosed based on the analysis of the removed portion of the interatrial septum tissue (228). Optionally, the patient may be notified of the diagnosis (230).

In some variations, one or more therapies may be administered to the patient based on the cardiac amyloidosis diagnosis (232). In some variations, the therapy may comprise one or more of a medication, chemotherapy, surgery, tissue ablation (e.g., using an ablation device), stent implantation, combinations thereof, and the like. For example, administering the therapy may comprise administering an effective amount of the medication comprising one or more of an immunosuppressive drug, steroid, tafamidis, diflunisal, patisiran, inotersen, combinations thereof, and the like, to the patient. In some variations, removing the portion of the interatrial septum and administering a therapy may separately or synergistically treat one or more of heart failure and cardiac amyloidosis.

In some variations, one or more therapies may be updated in response to the diagnosis. For example, the patient may be receiving therapy prior to performing the method (200) such that the analysis of captured septum tissue (226) may be used to update one or more of the diagnosis and therapy administered to the patient. For example, a lower dosage of medication may be administered in response to the updated septum tissue analysis.

In some variations, a method of treating a patient may include the step shown in FIG. 3A including advancing a tissue capture device (300) into a right atrium (330) of a patient. A distal end of the device (300) may comprise a dilator (350) configured to puncture an interatrial septum (310) and advance into a left atrium (320) of the patient. In some variations, a guidewire (not shown) of the device (300) may be advanced across the interatrial septum (310) and into the left atrium (320). As shown in FIG. 3B, the dilator (350) may puncture the septum (310) such that a distal portion of the tissue capture deice (300) is disposed within the left atrium (320) and a proximal portion (340) is disposed within the right atrium (330).

FIG. 3C illustrates the dilator (350) advanced relative to the proximal portion (340) such that a tissue engagement feature (360) is advanced across the septum (310) and into the left atrium (320). The tissue engagement feature (360) may be configured to engage a portion of the septum (310) for tissue separation and capture. For example, a portion of the engaged septum may be held and/or secured between the projections of the tissue engagement feature (360) (e.g., barb). By positioning the device (300) across both sides of the interatrial septum (310), a predetermined force may be applied from the proximal and distal portions (340, 350) to engage and cut a predetermined portion of septum tissue.

As shown in FIG. 3D, the distal portion (350) may be withdrawn relative to the proximal portion (340) such that a portion (312) of the septum (310) may engage the tissue engagement feature (360) (not shown in FIG. 3D) and stretch. The proximal portion (340) and the tissue engagement feature (360) may be positioned to engage opposite sides of the septum tissue (312). For example, withdrawal of the tissue engagement feature (360) into a lumen of the proximal portion (340) may engage and stretch the tissue (312) so as to form a tent-like shape that may aid formation of an anastomosis. Tissue (312) in FIG. 3D is shown tented towards the right atrium (330). In this manner, tissue (312) to be cut is secured within the device (300) prior to excision to reduce the risk of uncontrolled tissue loss in the heart chambers and vasculature.

In some of these variations, the proximal portion (340) of the tissue capture device (300) may comprise an electrode having a tubular shape configured to cut tissue using RF energy and promote tissue capture. An ablation waveform may be delivered to the electrode to cut the portion (312) of the interatrial septum (310) stretched by the device (300). For example, the ablation waveform may comprise RF energy as described in more detail herein.

Once the septum (310) is cut, as shown in FIG. 3E, a hole (314) may be formed in the septum (310). The distal portion (350) may be withdrawn from the left atrium (320) and the device (300) may be removed from the patient as shown in FIG. 3F. Accordingly, a tissue capture device (300) may form an interatrial anastomosis and capture an excised portion of tissue for analysis. The tissue capture devices as described herein may improve the efficacy and safety of an anastomosis procedure, as well as allow a reduction in a size of the device.

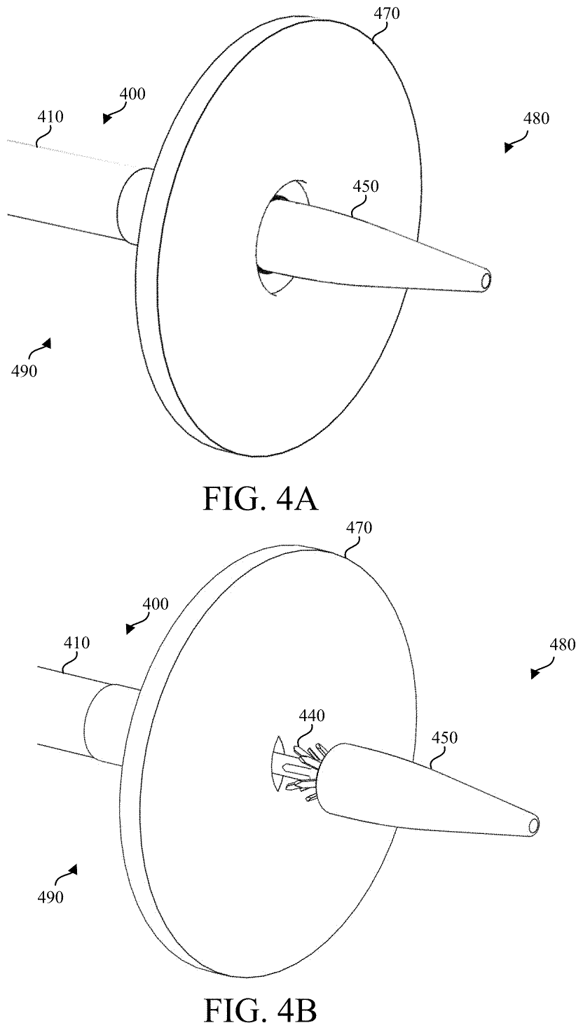

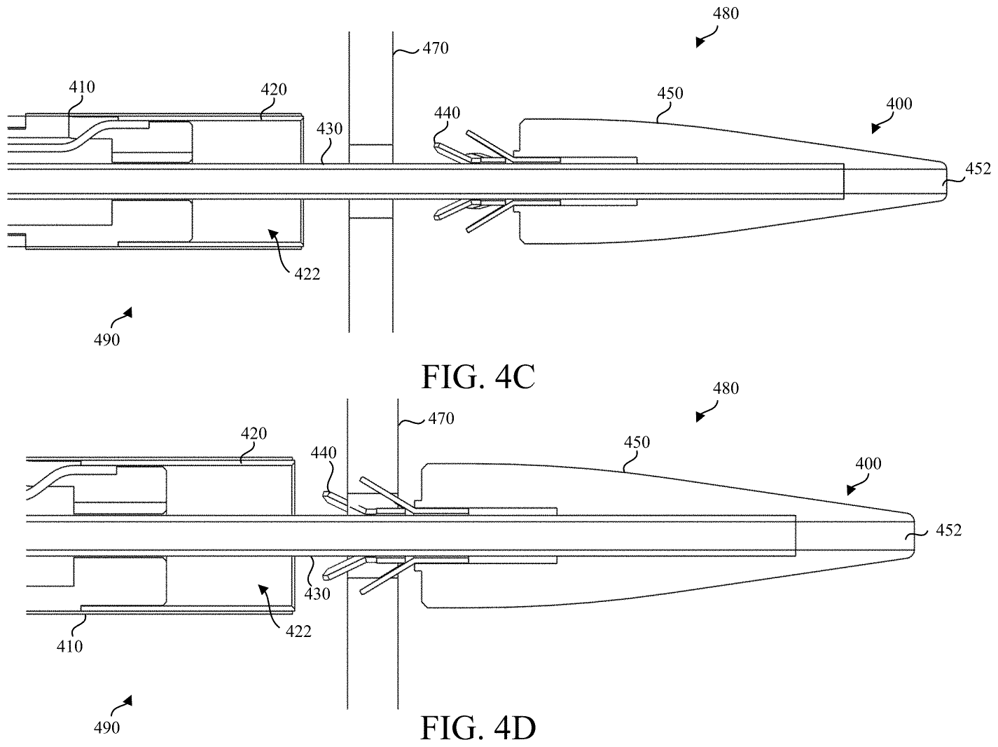

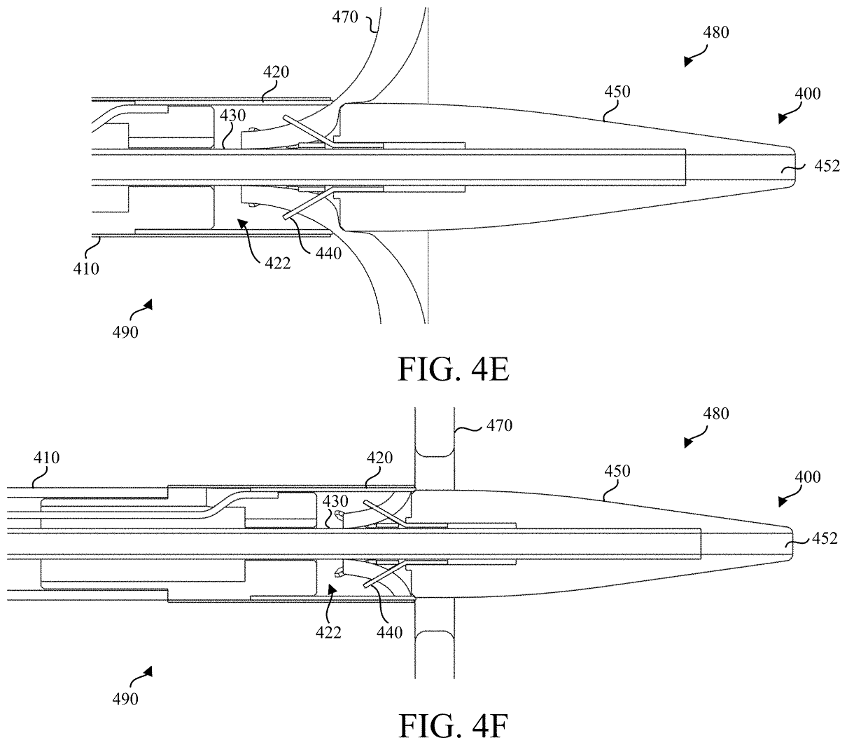

FIGS. 4A-4F illustrates another example of a patient treatment process. As shown in FIGS. 4A and 4B, a tissue capture device (400) may be disposed within a right atrium (490) and advanced into a left atrium (480) using a dilator of a second catheter (450). The second catheter (450) may be translated relative to a first catheter (410) in the right atrium (490) and the interatrial septum (470). The tissue engagement feature (440) of the second catheter (450) may be advanced through the septum (470) and into the left atrium (480). As shown in the cross-sectional side view of FIG. 4C, the first catheter (410) may comprise a tubular electrode (420), a lumen (422), a lead (424), connector (426), and insulator (460). The second catheter (450) may comprise a tissue engagement feature (440), mating surface (454), dilator, and dilator lumen (452).

As shown in FIG. 4D, as the second catheter (450) is withdrawn relative to the first catheter (410), the tissue engagement feature (440) may engage septum tissue (470). For example, the tissue engagement feature may pierce through the portion when withdrawing the second catheter towards the first catheter. The tissue engagement feature may pierce through the portion such that the layers of an interatrial septum (e.g., left and right atrium layers) are held together to reduce tissue separation and/or tissue shearing. Accordingly, the tissue engagement feature (440) may capture (e.g., secure, hold) tissue (470) while maintaining the structural integrity of the septum. In some variations, the withdrawn tissue engagement feature may apply a force to the septum to hold and stretch the portion of the septum over the tissue engagement feature. The force may increase as the second catheter is withdrawn further towards the first catheter. In some variations, withdrawal of the second catheter may apply a force of at least 20 grams to the interatrial septum. For example, the tissue capture device may apply a force of between about 20 grams and about 30 grams to the interatrial septum. In some variations, the portion of the septum may form a substantially cylindrical shape when withdrawn into the lumen.

The tissue engagement features described herein have a configuration designed to engage the portion of the septum without shearing the tissue (e.g., breaking or tearing through one or more layers of the interatrial septum) such that the portion remains intact when engaged to the tissue engagement feature and withdrawn into the lumen of the electrode. That is, the forces applied by the tissue engagement features described herein allow the structural integrity of the portion to be maintained even when the tissue engagement feature pierces through the septum. This may ensure that the portion of the septum to be excised remains held and secured by the tissue engagement feature throughout the procedure, thereby improving the consistency and safety of the methods described herein.

As shown in FIG. 4E, a portion of the septum (470) may form a tent-like shape over the tissue engagement feature (440). In some variations, the tissue engagement feature (470) engaged to tissue may rotate as it is withdrawn into the lumen of the electrode to apply a rotational force to the stretched (e.g., tented) septum tissue. In some variations, a size (e.g., diameter) of the tissue (470) to be cut may be controlled by varying a distance that the engaged tissue (470) is withdrawn into the lumen (422). Therefore, a size of an anastomosis may be independent of the electrode diameter. By withdrawing the second catheter towards the first catheter, the tissue capture device (400) engages, stretches, compresses, locks, and tents the tissue, as well as controls a size of the opening to be cut. In some variations, the size of an anastomosis may depend on the distance the tissue engagement feature is withdrawn into the electrode such that a size of an anastomosis may be independent of the diameter of the tissue capture device.

As shown in FIG. 4E, a portion of the interatrial septum (470) may be held between the electrode (420) and the dilator (450). For example, the electrode and the dilator may be brought together to abut (e.g., compress) opposite sides of the interatrial septum (470) to “lock” the tissue (470) in place relative to the tissue capture device (400). In some variations, the force applied to the interatrial septum by the tissue engagement feature (440) and through compression may be applied prior to and during delivery of the ablation waveform. The compressed tissue may allow a reduction in applied RF energy necessary to cut the tissue. In some variations, one or more of the tissue engagement feature and dilator may be rotated about a longitudinal axis of the second catheter to further engage and/or compress tissue.

FIG. 4F illustrates the interatrial septum (470) defining the predetermined opening and the tissue capture device (400) holding the excised tissue by the tissue engagement feature (440) within the lumen (422) of the electrode (420). As shown in FIG. 4F, the septum (470) may snap back after excising the tissue engaged by the tissue engagement feature (440). The tissue within the lumen (422) may be sealed within the tissue capture device (400) once tissue capture is completed and the electrode contacts the dilator (450). In this manner, excised tissue may be prevented from being lost in the body.

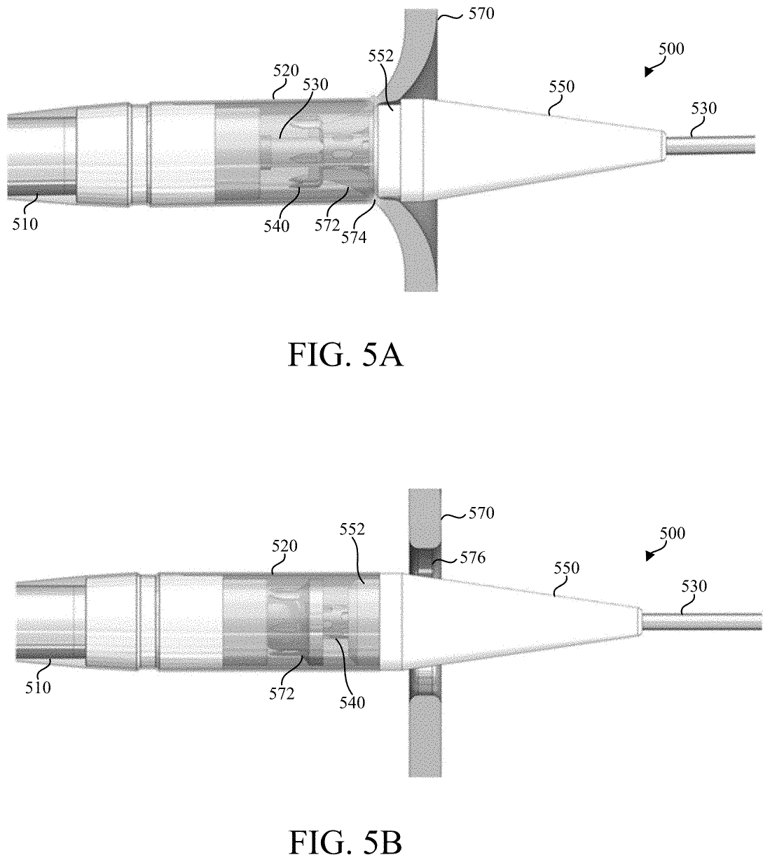

FIG. 5A is a side view of a tissue capture device (500) in an endocardial space illustrating compression step of a tissue capture procedure. In some variations, the tissue capture device (500) may comprise a first catheter (510), an electrode (520), second catheter (530), tissue engagement feature (540), and dilator (550). In some variations, the electrode (520) may comprise a lumen configured to hold one or more of the tissue engagement feature (540), a first portion (572) of tissue, and a proximal portion (552) of the dilator (550). In some variations, a guidewire (530) may be slidably disposed within the second catheter (530).

As depicted in FIG. 5A, the tissue engagement feature (540) may be configured to engage a first portion (572) of the interatrial septum (570) in a cutting configuration where the tissue (574) is compressed between a distal edge of the electrode (530) and the proximal portion (552) of the dilator (550). For example, a distal end of an electrode (520) may be configured to abut against a corresponding mating surface (552) of the dilator (550). For example, the second catheter (530) may be withdrawn with respect to the first catheter (510) such that a mating surface (552) applies a preload force to the tissue (574) and electrode (520). In some variations, application of the preload force may be controlled by an operator via an actuator of a handle. Compression of the tissue between the electrode and mating surface (via the preload force) may reduce the thickness of the tissue to be cut such that a septum may be cut faster and with less energy. Furthermore, compressed tissue may hold (e.g., secure, lock) the tissue in place relative to the tissue capture device to ensure that only a predetermined portion of tissue is cut. Compression of tissue may also reduce a volume of tissue. In some variations, a preload force may be between about 0.4 N to about 25 N, about 1 N to about 10 N, about 5 N to about 10 N, about 5 N to about 15 N, about 10 N to about 20 N, including all ranges and sub-values in-between.

In some variations, the compressed tissue (574) and the dilator (550) may come to rest in a static equilibrium state where the proximal portion (552) of the dilator (550) compresses the tissue (574) against the electrode (520) with a shear force comprising a radial component. In some variations, extension of the dilator (550) prior to cutting is beneficial to the operator when viewed fluoroscopically. In some variations, the tissue capture device (500) in the cutting configuration (FIG. 5A) may correspond to a dilator (550) being extended about 1 mm away from an end of the electrode (520).

FIG. 5B depicts the tissue capture device (500) in a closed (e.g., seated) configuration with cut tissue (e.g., first portion) (572) engaged to the tissue engagement feature (540) and held within a lumen of the electrode (520). The proximal portion (552) of the dilator (550) may be, for example, seated within the lumen of the electrode (520). FIG. 5B depicts a hole (576) formed in the interatrial septum (570).

In some variations, visualization may confirm the completion of an energy delivery process. For example, the differences between the tissue capture device (500) in the cutting configuration (FIG. 5A) and the closed configuration (FIG. 5B) may be confirmed through indirect visualization. For example, fluoroscopic visualization may confirm when tissue is interposed between the electrode (520) and dilator (550) and when tissue has been cut after energy delivery based on an imaged position of the dilator (550) relative to the electrode (520).

In some variations, a preload force (e.g., first predetermined force) may be applied by the dilator (550) to the electrode (520) during and/or after energy delivery to ensure withdrawal of the second catheter (530) towards the first catheter (510). In some variations, an operator may activate a switch in a handle to initiate energy delivery to cut tissue. As the proximal portion (552) withdraws toward and compresses against the electrode (520) during energy delivery, the proximal portion (552) may shear (e.g., cut, separate) the tissue from the septum (570) with a second predetermined force greater than the first predetermined force. That is, the proximal portion (552) may function as a cutting board to ensure that even small fibers of tissue (574) (e.g., second portion) are cut from the septum (570). Alternatively, a preload force may not be applied to the tissue (574) and electrode (520) when delivering an ablation waveform to the electrode (520). During energy delivery, the dilator (550) may naturally withdraw into the lumen of the electrode (520) after tissue (574) is cut (e.g., ablated).

In some variations, the tissue capture device (500) shown in FIG. 5B may be withdrawn from the patient. For each of the devices shown in FIG. 3A-5B, the tissue captured within the tissue capture devices may analyzed after withdrawal from the patient. One or more additional therapies may be administered to the patient based on the results of the captured tissue analysis, as described in more detail herein (e.g., FIG. 2).

### EXAMPLES

FIGS. 6A and 6B are images (600) of an anastomosis (620) formed in porcine tissue (610) using the tissue capture systems and methods described herein. The anastomosis (620) formed may be sufficient in size (e.g., have a diameter) to reduce blood pressure in a left atrium of a patient. In some variations, the captured tissue (e.g., removed tissue, cut tissue) may comprise a diameter of between about 1 mm and about 1.5 cm, including all ranges and sub-values in-between. For example, the captured tissue may comprise a diameter of between about 0.5 mm and about 12 mm. For example, the captured tissue may comprise a diameter of between about 6 mm and about 9 mm.

FIGS. 7A and 7B are images including a predetermined volume of captured tissue (750) that fits within a lumen (712) of an electrode (710). After the tissue capture device (700) is withdrawn from the patient, the captured tissue (750) may be separated from a tissue engagement feature (720) (e.g., barb) and analyzed to diagnose the patient and determine if administration of a another therapy is needed.

As yet another example, the captured tissue (850) shown in FIGS. 8A and 8B is engaged to a tissue engagement feature (810) (e.g., barb) where the captured tissue (850) includes a portion of the interatrial septum through an entire thickness of the interatrial septum (e.g., from a right atrium wall to a left atrium wall).

### II. System

Overview

Systems described here may include one or more of the components used to treat a patient using the devices as described herein. Generally, the systems described here may, for example, dispose portions of a tissue capture device on opposite sides of an interatrial septum. A portion of the septum may be engaged to the tissue capture device using one or more tissue engagement features (e.g., barb). In some variations, a portion of the tissue engagement feature may penetrate through the septum such that the tissue engagement feature may securely hold an intact portion of the septum tissue. The engaged tissue may be stretched, secured, and withdrawn into a lumen of the device for analysis outside the patient. In some variations, a size (e.g., diameter) of the tissue to be cut may be controlled by varying a distance that the engaged tissue is withdrawn into the lumen.

An optional electrode may use radiofrequency (RF) energy to ablate tissue to form an anastomosis in the interatrial septum. Additionally or alternatively, a blade or other cutting mechanism may be configured to cut a portion of the interatrial septum from the heart. After ablation and/or cutting, the portion of tissue engaged, held, and/or secured by the tissue engagement feature may remain enclosed within the lumen of the device for removal from the patient. One or more steps of a treatment procedure may be visualized using one or more visualization techniques and visualization features incorporated within the tissue capture device. Accordingly, the tissue capture device as described herein may improve the efficacy and safety of an anastomosis formation procedure, as well as allow a size of the catheter to be reduced.

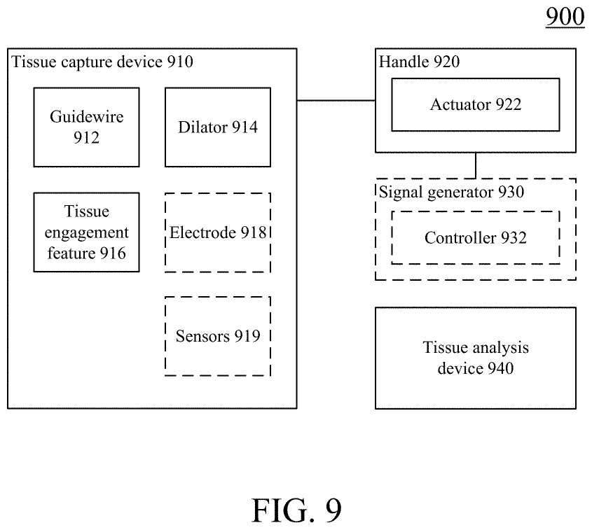

FIG. 9 is a block diagram of a variation of a tissue capture system (900) comprising a tissue capture device (910), handle (920), optional signal generator (930), and tissue analysis device (940). In some variations, the tissue capture device (910) may be designed to be disposable after each use, while in other variations, one or more portions of the tissue capture device (910) may be designed to be reusable (e.g., used multiple times, and with one or more patients) such as the handle (920), signal generator (930), and tissue analysis device (940).

In some variations, the tissue capture device (910) may be sized and shaped to be placed in a body cavity of the patient such as a heart chamber. In some variations, the tissue capture device (910) may comprise one or more of a guidewire (912), dilator (914), tissue engagement feature (916), and optional electrode (918). A distal portion of the tissue capture device (910) may comprise the dilator (914) and the guidewire (912) may extend from a lumen of the dilator (914). In some variations, an optional electrode (918) may be disposed proximal to the tissue engagement feature (916), while in other variations, the electrode (918) may be disposed distal to the tissue engagement feature (918). Additionally or alternatively, the tissue capture system (900) may comprise a delivery catheter configured to advance over the tissue capture device (910). Moreover, the tissue capture device (910) may comprise one or more optional sensors (919) configured to measure one or more predetermined characteristics such as temperature, pressure, impedance, and the like.

In some variations, a proximal end of the tissue capture device (910) may be coupled to a handle (920). The handle (920) may comprise an actuator (922) configured to control one or more of movement, positioning, configuration, orientation, operation, and energy delivery of the tissue capture device (910). For example, the actuator (922) may be operated to steer and/or translate one or more portions of the tissue capture device (910). In some variations, the actuator (922) may be configured to mechanically cut (e.g., excise) tissue. In some variations, a signal generator (930) may be coupled to one or more of the tissue capture device (910) and handle (920). The signal generator (930) may be configured to generate one or more ablation waveforms for delivery to the electrode (918) of the tissue capture device (910). The signal generator (930) may comprise a controller (932) configured to control the signal generator (930) and provide appropriate energy waveforms for tissue ablation and ensure patient safety.

In some variations, a tissue analysis device (940) may be configured to analyze the captured portion of tissue (e.g., interatrial septum tissue). For example, the tissue analysis device (940) may comprise one or more devices configured for one or more of histological analysis, mass spectrometry, and genetic testing. For example, the tissue analysis device (940) may be configured to stain (e.g., Congo red) the captured interatrial septum tissue and enable histological analysis and diagnosis of cardiac amyloidosis.

In some variations, the system and devices described herein may comprise one or more features of the systems and devices described in U.S. application Ser. No. 16/533,655, filed on Aug. 6, 2019, and titled “Transcatheter Device for Interatrial Anastomosis,” and U.S. application Ser. No. 16/886,467, filed on May 28, 2020, and titled “Transcatheter Device for Interatrial Anastomosis,” and U.S. application Ser. No. 17/019,042, filed on Sep. 11, 2020, and titled “Systems, Devices, and Methods for Forming an Anastomosis,” each of which is hereby incorporated by reference in its entirety.

FIG. 10 is a schematic cross-sectional side view of a variation of a tissue capture device (1000) in an open configuration. In some variations, the tissue capture device (1000) may comprise a first catheter (1010) (e.g., proximal portion) and a second catheter (1030) (e.g., distal portion). The first catheter (1010) may comprise an electrode (1020) such as a tubular electrode. The electrode (1020) may define a lumen configured to hold one or more portions of the second catheter (1030). The first catheter (1010) may further comprise a first catheter actuator (1022) (e.g., lead, electrical pull wire) coupled to the electrode (1020) and a signal generator (not shown). As described in more detail herein, the first catheter actuator (1022) may be configured to deliver electrical energy to the electrode (1020) as well as deflect a distal portion of the tissue capture device (1000) in the manner of a pull wire. In some variations, the first catheter (1010) may comprise an insulator (1024) configured to cover a portion of the electrode (1020). For example, the insulator (1024) may be configured to cover an outer surface of the electrode (1020) where a distal end and an inner surface of the electrode (1020) are uninsulated.

In some variations, the tissue capture device (1000) may comprise a second catheter (1030) slidably disposed within the first catheter (1010). The second catheter (1030) may be configured to translate relative to the first catheter (1010) via an actuation mechanism of a handle. The second catheter (1030) may comprise a tissue engagement feature (1040) and a dilator (1050) configured to engage the electrode (1020). In some variations, the tissue engagement feature (1040) may comprise a plurality of projections (1042, 1044) radially arranged that generally extend towards the electrode (1020). For example, the projections (1042, 1044) may comprise a distal portion (1042) configured to pierce tissue and a proximal portion (1044) configured as a backstop to tissue.

In some variations, the dilator (1050) may be tapered and define a lumen (1052). In some variations, a guidewire (not shown) may be slidably disposed within the lumen (1052). In some variations, the dilator (1050) may comprise a proximal portion (1054). The proximal portion (1054) of the dilator (1050) may be configured to engage the electrode (1020) in the closed configuration. That is, the proximal portion (1054) may be configured to be in a lumen of the electrode (1020) in the closed configuration of the tissue capture device (1000). In some variations, the dilator (1050) may comprise a mating surface (1056) configured to engage the electrode (1020). For example, the electrode (1020) and mating surface (1056) may be configured to compress tissue therebetween (not shown).

In the closed configuration (not shown), the tissue engagement feature (1040) may be enclosed by the first catheter (1010), electrode (1020), and dilator (1050). That is, the tissue engagement feature (1040) may be disposed within a lumen of the electrode (1020) when the proximal portion (1054) (e.g., mating surface (1056)) engages (e.g., is seated within) the electrode (1020). Accordingly, any tissue engaged by the tissue engagement feature (1040) may also be captured (e.g., enclosed, secured) within the tissue capture device (1000) in the closed configuration by one or more of the tissue engagement feature (1040) and electrode (1020).

### Electrode

Generally, the electrodes described here may be configured to ablate tissue such as a portion of an interatrial septum of a patient to reduce blood pressure in a left atrium of a patient. In some variations, the electrode may engage the septum and be energized to excise a portion of septum tissue to form a predetermined opening between the left atrium and right atrium. For example, tissue may be heated using radiofrequency (RF) energy during an electrosurgical procedure. RF energy tissue ablation may be used to quickly and precisely cut tissue without significant damage to surrounding tissue. In some variations, RF energy may be delivered to tissue by an electrode to quickly and precisely cut tissue so as to form an anastomosis of a predetermined shape and size.

In some variations, tissue ablation characteristics may be controlled by the size, shape, and/or geometry of the conductive region of the electrode. In some variations, the electrode may comprise one or more biocompatible metals such as titanium, stainless steel, nitinol, palladium, silver, platinum, combinations thereof, and the like. In some variations, the electrode may comprise an atraumatic (e.g., blunt, rounded) distal edge such that the electrode does not puncture tissue when pressed against an opposing surface such as a mating surface of a dilator. For example, the electrode may engage and compress the tissue along its chamfered circumferential edge.

In some variations, the cut tissue may comprise a diameter of between about 1 mm and about 1.5 cm, including all ranges and sub-values in-between. For example, the cut tissue may comprise a diameter of between about 0.5 mm and about 12 mm. For example, the cut tissue may comprise a diameter of between about 6 mm and about 9 mm.

### Tissue Engagement Feature

Generally, the tissue engagement features (e.g., barb, arm, leg, projection) described here may be configured to engage tissue such as an interatrial septum of a patient to control a size and shape of the septum tissue to be cut. In some variations, a portion of septum tissue may be engaged and stretched across the tissue engagement features to hold tissue in place before and after tissue capture. In some variations, the tissue engagement features may be configured to penetrate a predetermined distance into the tissue or through the tissue. For example, the tissue engagement features may be configured to penetrate through multiple layers of the interatrial septum (e.g., one or more left atrium layers and right atrium layers) to secure the septum tissue to the tissue engagement feature while maintaining the structural integrity of the septum as a whole. In some of these variations, penetration through the tissue may hold the tissue to the tissue engagement feature to reduce the shearing strain of the tissue as it is captured, thereby improving the consistency and shape (e.g., cylindricity) of the cut. That is, the tissue engagement feature may be configured to capture but not tear tissue such that the tissue may remain engaged by the tissue engagement feature throughout an electrosurgical procedure. In some variations, a tissue engagement feature may comprise one or more of a spiral, helical, corkscrew, and coil shape.

In some variations, the tissue engagement feature may be formed to have enough strength to hold tissue without breaking. The tissue engagement feature may comprise one or more of stainless steel, nitinol, platinum, polyvinyl chloride (PVC), polyethylene (PE), cross-linked polyethylene, polyolefins, polyolefin copolymer (POC), polyethylene terephthalate (PET), polyester, nylon, polymer blends, polyester, polyimide, polyamides, polyurethane, silicone, polydimethylsiloxane (PDMS), PEBAX, combinations thereof, and the like.

### Guidewire

In some variations, a guidewire may be slidably disposed within a tissue capture device and configured to cross the interatrial septum (e.g., using a standard transseptal puncture technique). In some variations, the tissue capture device may be translated along the guidewire relative to one another and/or the interatrial septum. For example, the guidewire may comprise one or more of stainless steel, nitinol, platinum, and other suitable, biocompatible materials.

### Catheter

Generally, the catheters described here may be configured to deliver an electrode and tissue engagement feature to one or more heart chambers for cutting tissue such as an interatrial septum. In some variations, a catheter may comprise a shaft composed of a flexible polymeric material such as Teflon, Nylon, Pebax, combinations thereof, and the like. In some variations, the tissue capture device may comprise one or more steerable or deflectable catheters (e.g., unidirectional, bidirectional, 4-way, omnidirectional). In some variations, the first catheter may comprise one or more pull wires configured to steer or deflect a portion of the first catheter. In some variations, the first catheter may have a bend radius of between about 45 degrees and about 270 degrees. In some variations, the second catheter described herein define a lumen through which a guidewire may pass.

In some variations, the catheter may be woven and/or braided and composed of a material (e.g., nylon, stainless steel, polymer) configured for catheter pushability and flexibility. In some variations, a first catheter may comprise a predetermined curved shape configured to guide a second catheter towards the septum at a predetermined orientation and angle.

### Dilator

Generally, the dilators described here may be configured to puncture tissue such as an interatrial septum to allow one or more portions of a tissue capture device to be advanced into a body cavity such as a left atrium of the heart. In some variations, a dilator may generally be configured to dilate tissue such as an interatrial septum. The dilator may be atraumatic in profile to minimize any inadvertent or unintended damage. The dilator may comprise a taper of between about 1 degree and about 45 degrees to facilitate device crossing of the septum to the left atrium. In some variations, the dilator may comprise a thermoplastic polymer, nylon, polyurethane, ABS, acetal, polycarbonate, PET, PEBA, PEEK, PTFE, silicone, PS, PEI, latex, sulphate, barium sulfate, a copolymer, combinations thereof, and the like. As described in more detail herein, a dilator may comprise one or more visualization features such as an echogenic region.

### Handle

Generally, the handles described here may be configured to allow an operator to grasp and control one or more of the position, orientation, and operation of a tissue capture device. In some variations, a handle may comprise an actuator to permit translation and/or rotation of the tissue capture device in addition to steering by an optional delivery catheter. Deployment of a tissue engagement feature, in some variations, may be performed by a deployment mechanism (e.g., screw/rotation mechanism, translation mechanism, slider). In some variations, the handle may be configured to limit the applied force that a user may administer to the advancement and retraction of the catheter shafts relative to each other. For example, the handle may be configured to apply energy to the electrode to ablate tissue and/or control one or more sensors. In some variations, the handle may be coupled between a signal generator and a tissue capture device.

### Signal Generator

Generally, the signal generators described here may be configured to provide energy (e.g., energy waveforms) to a tissue capture device to ablate predetermined portions of tissue such as an interatrial septum. In some variations, a tissue capture system as described herein may include a signal generator having an energy source and a processor configured to deliver a waveform to deliver energy to tissue (e.g., interatrial septum). The waveforms disclosed herein may aid in forming an anastomosis. In some variations, the signal generator may be configured to control waveform generation and delivery in response to received sensor data. For example, energy delivery may be inhibited unless a pressure sensor measurement confirms tissue engagement and compression between an electrode and corresponding mating surface.

The signal generator may generate and deliver several types of signals including, but not limited to, radiofrequency (RF), direct current (DC) impulses, stimulus range impulses, and/or hybrid electrical impulses. For example, the signal generator may generate monophasic (DC) pulses and biphasic (DC and AC) pulses. The signal generator may comprise a processor, memory, energy source, and user interface. The processor may incorporate data received from one or more of memory, energy source, user interface, tissue capture device. The memory may further store instructions to cause the processor to execute modules, processes and/or functions associated with the system, such as waveform generation and delivery. For example, the memory may be configured to store patient data, clinical data, procedure data, and the like.

In some variations, the signal generator may be configured to generate alternating current, voltage, and/or power in the radiofrequency spectrum between about 9 kHz and about 300 MHz at a power level between about 5 W and about 500 W. In some variations, the RF generator may be operated by outputting constant voltage, constant power, and/or constant current. In some variations, the RF generator may be configured to output a constant sine wave throughout the duration of tissue cutting. For example, the RF generator may be configured to output a sine wave between about 400 kHz and about 600 kHz, between about 450 kHz and about 550 kHz, and between about 475 kHz and about 525 kHz, including all values and sub-ranges in-between. In some variations, the RF signal output may be interrupted and dampened such that RF energy is applied for a fixed percentage of operation time.

In some variations, the signal generator may be configured to synchronize energy delivery with a predetermined phase of a patient's cardiac cycle. For example, a sensor may be configured to measure an ECG signal and the signal generator may be configured to deliver a signal waveform based on (e.g., in synchronicity) with the ECG signal. Additionally or alternatively, a pacing signal for cardiac stimulation may be generated and used to deliver a signal waveform by the signal generator in synchronization with the pacing signal.

Generally, the processor (e.g., CPU) described here may process data and/or other signals to control one or more components of the system. The processor may be configured to receive, process, compile, compute, store, access, read, write, and/or transmit data and/or other signals. In some variations, the processor may be configured to access or receive data and/or other signals from one or more of a sensor (e.g., pressure sensor) and a storage medium (e.g., memory, flash drive, memory card). In some variations, the processor may be any suitable processing device configured to run and/or execute a set of instructions or code and may include one or more data processors, image processors, graphics processing units (GPU), physics processing units, digital signal processors (DSP), analog signal processors, mixed-signal processors, machine learning processors, deep learning processors, finite state machines (FSM), compression processors (e.g., data compression to reduce data rate and/or memory requirements), encryption processors (e.g., for secure wireless data and/or power transfer), and/or central processing units (CPU). The processor may be, for example, a general purpose processor, Field Programmable Gate Array (FPGA), an Application Specific Integrated Circuit (ASIC), a processor board, and/or the like. The processor may be configured to run and/or execute application processes and/or other modules, processes and/or functions associated with the system. The underlying device technologies may be provided in a variety of component types (e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and/or the like.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

Generally, the tissue capture device described here may comprise a memory configured to store data and/or information. In some variations, the memory may comprise one or more of a random access memory (RAM), static RAM (SRAM), dynamic RAM (DRAM), a memory buffer, an erasable programmable read-only memory (EPROM), an electrically erasable read-only memory (EEPROM), a read-only memory (ROM), flash memory, volatile memory, non-volatile memory, combinations thereof, and the like. In some variations, the memory may store instructions to cause the processor to execute modules, processes, and/or functions associated with a tissue capture device, such as signal waveform generation, tissue capture device control, data and/or signal transmission, data and/or signal reception, and/or communication. Some variations described herein may relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes.

In some variations, the tissue capture device may further comprise a communication device configured to permit an operator to control one or more of the devices of the tissue capture system. The communication device may comprise a network interface configured to connect the tissue capture device to another system (e.g., Internet, remote server, database) by wired or wireless connection. In some variations, the tissue capture device may be in communication with other devices (e.g., cell phone, tablet, computer, smart watch, and the like) via one or more wired and/or wireless networks. In some variations, the network interface may comprise one or more of a radiofrequency receiver/transmitter, an optical (e.g., infrared) receiver/transmitter, and the like, configured to communicate with one or more devices and/or networks. The network interface may communicate by wires and/or wirelessly with one or more of the tissue capture device, network, database, and server.

The network interface may comprise RF circuitry configured to receive and/or transmit RF signals. The RF circuitry may convert electrical signals to/from electromagnetic signals and communicate with communications networks and other communications devices via the electromagnetic signals. The RF circuitry may comprise well-known circuitry for performing these functions, including but not limited to an antenna system, an RF transceiver, one or more amplifiers, a tuner, one or more oscillators, a mixer, a digital signal processor, a CODEC chipset, a subscriber identity module (SIM) card, memory, and so forth.

Wireless communication through any of the devices may use any of plurality of communication standards, protocols and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol. In some variations, the devices herein may directly communicate with each other without transmitting data through a network (e.g., through NFC, Bluetooth, WiFi, RFID, and the like).

In some variations, the user interface may comprise an input device (e.g., touch screen) and output device (e.g., display device) and be configured to receive input data from one or more of the tissue capture device, network, database, and server. For example, operator control of an input device (e.g., keyboard, buttons, touch screen) may be received by the user interface and may then be processed by processor and memory for the user interface to output a control signal to the tissue capture device. Some variations of an input device may comprise at least one switch configured to generate a control signal. For example, an input device may comprise a touch surface for an operator to provide input (e.g., finger contact to the touch surface) corresponding to a control signal. An input device comprising a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In variations of an input device comprising at least one switch, a switch may comprise, for example, at least one of a button (e.g., hard key, soft key), touch surface, keyboard, analog stick (e.g., joystick), directional pad, mouse, trackball, jog dial, step switch, rocker switch, pointer device (e.g., stylus), motion sensor, image sensor, and microphone. A motion sensor may receive operator movement data from an optical sensor and classify an operator gesture as a control signal. A microphone may receive audio data and recognize an operator voice as a control signal.

A haptic device may be incorporated into one or more of the input and output devices to provide additional sensory output (e.g., force feedback) to the operator. For example, a haptic device may generate a tactile response (e.g., vibration) to confirm operator input to an input device (e.g., touch surface). As another example, haptic feedback may notify that operator input is overridden by the tissue capture device.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within ±10% of the recited value. For example, in some instances, “about 100 [units]” may mean within ±10% of 100 (e.g., from 90 to 110). The terms “about” and “approximately” may be used interchangeably.

The specific examples and descriptions herein are exemplary in nature and variations may be developed by those skilled in the art based on the material taught herein without departing from the scope of the present invention, which is limited only by the attached claims.

Although the foregoing implementations has, for the purposes of clarity and understanding, been described in some detail by of illustration and example, it will be apparent that certain changes and modifications may be practiced, and are intended to fall within the scope of the appended claims. Additionally, it should be understood that the components and characteristics of the elements described herein may be used in any combination, and the methods described herein may comprise all or a portion of the elements described herein. The description of certain elements or characteristics with respect to a specific figure are not intended to be limiting or nor should they be interpreted to suggest that the element cannot be used in combination with any of the other described elements.

In addition, any combination of two or more such features, structure, systems, articles, materials, kits, steps and/or methods, disclosed herein, if such features, structure, systems, articles, materials, kits, steps and/or methods are not mutually inconsistent, is included within the inventive scope of the present disclosure. Moreover, some variations disclosed herein may be distinguishable from the prior art for specifically lacking one or more features, elements, and functionality found in a reference or combination of references (i.e., claims directed to such variations may include negative limitations).

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

We claim:

1. A method, comprising:
removing a portion of an interatrial septum; and
analyzing the removed portion for cardiac amyloidosis.

2. A method of treating a patient, comprising:
removing a portion of an interatrial septum;
diagnosing cardiac amyloidosis based on analyzing the removed portion of the interatrial septum; and
administering a therapy to the patient based on the cardiac amyloidosis diagnosis.

3. The method of any one of claims 1 and 2, wherein analyzing the removed portion of the interatrial septum comprises one or more of histological analysis, electron microscopy, mass spectrometry, and genetic testing.

4. The method of any one of claims 1 and 2, wherein analyzing the removed portion of the interatrial septum comprises analysis for one or more of fibrosis, hypertrophy, and inflammation.

5. The method of any one of claims 1 and 2, wherein the therapy comprises one or more of a medication, chemotherapy, surgery, and stent implantation.

6. The method of claim 2, wherein administering the therapy comprises administering an effective amount of the medication comprising one or more of an immunosuppressive drug, steroid, tafamidis, diflunisal, patisiran, and inotersen to the patient.

7. The method of any one of claims 1 and 2, further comprising removing a portion of right ventricle tissue, and analyzing the removed portion of the right ventricle tissue for the cardiac amyloidosis diagnosis.

8. The method of claim 2, wherein removing the portion of the interatrial septum and administering the therapy treats one or more of heart failure and cardiac amyloidosis.

9. The method of any one of claims 1 and 2, wherein removing the portion of the interatrial septum comprises one or more of mechanical cutting and tissue ablation using a tissue capture device.

10. The method of claim 9, wherein the anastomosis is formed using the tissue capture device.

11. The method of claim 9, wherein the tissue ablation comprises delivering a biphasic radiofrequency waveform to the interatrial septum.

12. The method of any one of claims 1 and 2, further comprising:
advancing a tissue capture device into a heart of the patient;
capturing the portion of the interatrial septum using the tissue capture device; and
withdrawing the portion of the interatrial septum and the tissue capture device from the heart.

13. The method of claim 12, wherein the captured portion of the interatrial septum is disposed in a lumen of the tissue capture device.

14. The method of claim 1, wherein the anastomosis comprises a diameter of between about 1 mm and about 1.5 cm.

15. The method of any of claims 1 and 2, wherein removing the portion of the interatrial septum forms an anastomosis between a right atrium and a left atrium of the patient.

16. The method of any one of claims 1 and 2, wherein removing the portion of the interatrial septum reduces blood pressure in a left atrium of the patient.
